(19) United States
(12) Patent Application Publication
(10) Pub. No.: US 2015/0087681 A1
(43) Pub. Date: Mar. 26, 2015

(54) Bendamustine HCL Stable Lyophilized Formulations

(71) Applicants: Pranav Patel, East Windsor, NJ (US); Mahendra R. Patel, Milltown, NJ (US)

(72) Inventors: Pranav Patel, East Windsor, NJ (US); Mahendra R. Patel, Milltown, NJ (US)

(21) Appl. No.: 14/496,893

(22) Filed: Sep. 25, 2014

Related U.S. Application Data
(60) Provisional application No. 61/882,328, filed on Sep. 25, 2013.

(51) Int. Cl. A61K 47/12; A61K 47/10; A61K 9/19; A61K 31/4184

(52) U.S. Cl. CPC A61K 47/12; A61K 31/4184; A61K 47/10; A61K 9/19
USPC 514/394

(57) ABSTRACT

The present invention provides a lyophilized bendamustine hydrochloride (HCL) pharmaceutical composition. The present invention further provides methods of producing the lyophilized bendamustine HCL composition from a composition including bendamustine HCL, mannitol, formic acid, and water. The pharmaceutical formulation can be used for any disease that is sensitive to treatment with bendamustine, such as neoplastic diseases.

### FIELD OF THE INVENTION

The present relates to a pharmaceutical formulation of bendamustine or a pharmaceutically acceptable salt thereof.

### BACKGROUND OF THE INVENTION

Bendamustine is one species of nitrogen mustards. It has the chemical name: 4[5-[bis(2-chloroethyl)amino]-1-methylbenzimidazol-2-yl]butanoic acid, with the following structure (Formula I):

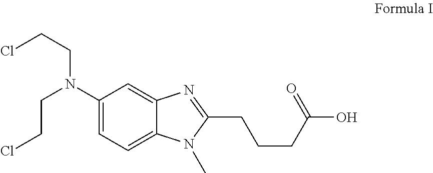 [SMILES: Cn1c(CCCC(=O)O)nc2cc(N(CCCl)CCCl)ccc21]

Bendamustine was initially synthesized in 1963 in the German Democratic Republic. Bendamustine received its first marketing approval in Germany, where it is marketed under the tradename Ribomustin®. It was indicated as a single-agent or in combination with other anti-cancer agents for a number of cancers including leukemia, Hodgkin's disease, and multiple myelomas. Bendamustine is the active ingredient of the commercial drug product Treanda®, a lyophilized powder for reconstitution. Treanda® is approved by U.S. FDA in 2008 for the treatment of chronic lymphocytic leukemia (CLL) and indolent B-cell non-Hodgkin lymphoma (NHL) that has progressed during or within six months of treatment with rituximab or a rituximab-containing regimen.

Bendamustine is a white to off-white, water soluble microcrystalline powder with amphoteric properties. Bendamustine is not stable in water. In aqueous solutions, bendamustine rapidly hydrolyzes by direct substitution, leading to three main degradation impurities: a monohydroxy compound (Formula II) (the main degradant), a dihydroxy compound (Formula III), and rarely, a dimer compound (Formula IV), with the following structures:

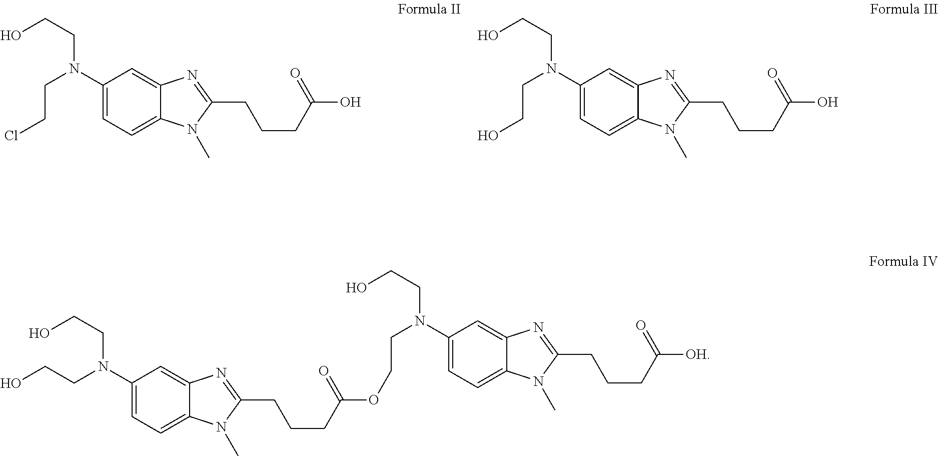 [SMILES: Cn1c(CCCC(=O)O)nc2cc(N(CCO)CCCl)ccc21 ; Cn1c(CCCC(=O)O)nc2cc(N(CCO)CCO)ccc21 ; Cn1c(CCCC(=O)O)nc2cc(N(CCO)CCOC(=O)CCCc3nc4cc(N(CCO)CCO)ccc4n3C)ccc21]

For this reason, bendamustine is not suitable for long-term storage in an aqueous solution form. The lyophilized bendamustine has very good chemical stability. However, upon reconstitution of the lyophilate, bendamustine undergoes rapid degradation, producing substantially the same main degradants. Therefore, the commercial product, Treanda®, is supplied as a sterile non-pyrogenic lyophilized powder in a single-use sealed vial. Each 25-mg vial contains 25 mg of bendamustine hydrochloride and 42.5 mg of mannitol, USP. Each 100-mg vial contains 100 mg of bendamustine hydrochloride and 170 mg of mannitol, USP. Prior to use, each of the 25-mg vial and 100-mg vial is opened and reconstituted with 5 mL or 20 mL of Sterile Water for Injection, USP, and further diluted with either 0.9% Sodium Chloride Injection, USP, or 2.5% Dextrose/0.45% Sodium Chloride Injection, USP, to form a reconstituted solution with the concentration of bendamustine HCL within 0.2 mg/mL-0.6 mg/mL. The reconstituted solution has to be administrated to the patient as soon as possible. Any unused solution should be discarded according to institutional procedures for antineoplastics. (See, http://dailymed.nlm.nih.gov/dailymed/lookup.cfm?setid=39d53698-57fa-7c99-fc5b-f52a55684826#section-3, drug label for “TREANDA (bendamustine hydrochloride) injection, powder, lyophilized, for solution”).

Numerous literatures disclose the preparation of bendamustine, its pharmaceutically acceptable salts, and compositions thereof. German Patent No. 159877 (GDR) discloses a method for preparing 4-[1-methyl-5-bis(2-chloroethyl)amino-benzimidazolyl-2)-butyric acid. German Patent No. 159289 discloses details of an injectable solution of bendamustine.

Lyophilized bendamustine compositions are disclosed in U.S. Pat. Nos. 8,436,190, 8,461,350, 8,609,863 and 8,791,270, of the same patent family, the teachings of which are incorporated herein by reference. The patents provide methods of producing lyophilized bendamustine compositions suitable for pharmaceutical drug uses. The methods comprise the step of lyophilizing a pharmaceutical composition containing bendamustine or bendamustine hydrochloride, mannitol, water, and a solvent selected from ethanol, n-propanol, n-butanol, t-butanol (a.k.a., tert-butyl alcohol, or TBA), isopropanol, methanol, ethyl acetate, dimethyl carbonate, acetonitrile, dichloromethane, methyl ethyl ketone, methyl isobutyl ketone, acetone, 1-pentanol, methyl acetate, carbon tetrachloride, dimethyl sulfoxide, hexafluoroacetone, chlorobutanol, dimethyl sulfone, acetic acid, cyclohexane, and a combination thereof. However, the patents in fact only teach 30% TBA in water as the only solvent system that produces an acceptable lyophilate which will reconstitute within 3-5 minutes. Reconstitution of a lyophilate from other solvent systems is difficult and may take more than 45 minutes.

But the lyophilization method using a composition having 30% TBA is still not ideal. First, the lyophilized pharmaceutical compositions may contain a trace amount of t-butanol and up to 0.9% of the monohydroxy degradation byproduct upon reconstitution. Moreover, the lyophilized pharmaceutical compositions may additionally contain bendamustine ester as an impurity.

Therefore, there still exists a need for lyophilized bendamustine formulations that are easier to reconstitute, have better stability and improved impurity profiles than the lyophilized powder of bendamustine currently on the market.

### SUMMARY OF THE INVENTION

The present invention provides a novel bendamustine pharmaceutical composition comprising bendamustine or bendamustine HCL, mannitol, formic acid, and water. In one embodiment of the pharmaceutical composition, bendamustine or bendamustine hydrochloride is present at a concentration of about 5 mg/mL to about 20 mg/mL, mannitol is present at a concentration of about 10 mg/mL to about 30 mg/mL, formic acid is present at a concentration of about 5% (v/v) to about 70% (v/v), and water constitutes the rest of the pharmaceutical composition.

The bendamustine pharmaceutical composition is suitable for lyophilization to provide a lyophilized bendamustine composition with an improved impurity and stability profile. According to one embodiment of the lyophilized bendamustine composition, the composition contains not more than 1.0%, preferably not more than 0.5%, of bendamustine monohydroxy impurity upon reconstitution at time zero. According to another embodiment, the lyophilized bendamustine composition contains not more than 0.5%, preferably from not more than 0.2% of bendamustine dihydroxy impurity.

The present invention also provides a novel and stable process for the preparation of a bendamustine formulation that controls bendamustine degradation impurities. The total bendamustine impurities in the final product are less than 3.0%.

The process comprises preparing a pharmaceutical composition (a.k.a., pre-lyophilization composition) having bendamustine or bendamustine hydrochloride, mannitol, formic acid, and water, and then subjecting the pharmaceutical composition to lyophilization. The process may further comprises the step of reconstituting the lyophilized pharmaceutical composition followed by diluting the resulting solution.

The present invention further provides a method of treating neoplastic diseases (i.e., cancers) in mammals by administering an effective amount of a pharmaceutical composition of bendamustine or bendamustine hydrochloric acid to a mammal in need thereof.

### DETAILED DESCRIPTION OF THE INVENTION

Unless defined otherwise, all technical and scientific terms used herein have the same meaning as is commonly understood by one of ordinary skill in the art to which this invention belongs. In the event that there is a plurality of definitions for a term herein, those defined in the specification or incorporated by reference prevail.

In accordance with one aspect of the invention, there is provided a pharmaceutical composition comprising bendamustine or bendamustine hydrochloride, mannitol, formic acid, and water.

A novel feature of the pharmaceutical composition is the inclusion of formic acid. Formic acid is one of the safe solvents for pharmaceutical use. (See International Conference on Harmonisation (“ICH”), class III, CPMP/ICH/283/95 dated February, 2009, section 4.3, solvents with low toxic potential). Diluted formic acid is on the U.S. FDA list of food additives. Formic acid is readily metabolized and eliminated by the body.

Additionally, formic acid is miscible with water in all the proportions (v/v). The co-solvents of formic acid and water allow mannitol and bendamustine to fully dissolve in the solvent mixture without difficulty. This is an advantage over the prior art that uses alcohol as a co-solvent of water in preparing bendamustine compositions. It is reported that mannitol decreases the solubility of bendamustine (at 15 mg/mL) in both ethanol and TBA aqueous solutions. However, mannitol is required in order to maintain a stable bendamustine pharmaceutical formulation similar to Treanda®.

Formic acid, when added to the bendamustine composition, not only improves solubility of the composition, but also stabilizes bendamustine contained therein. The essential components of the pharmaceutical compositions include bendamustine, mannitol, formic acid, and water.

Pure formic acid is a colorless fuming liquid. For the ease of handling, a formic acid solution in water (i.e., formic acid aqueous solution) is typically used for preparing the composition. Formic acid aqueous solutions are commercially available at various concentrations. Preferably, concentrated formic acid aqueous solutions are used. The term “concentrated formic acid aqueous solution” means that the formic acid concentration in water is from about 80% (v/v) to about 90% (v/v).

Regardless what concentration of a commercial formic acid aqueous solution is used during the process, the final formic acid concentration in the pharmaceutical composition is preferably about 5% (v/v) to about 70% (v/v), more preferably about 10% (v/v) to about 60% (v/v), and even more preferably from about 10% (v/v) to about 30% (v/v).

Bendamustine or bendamustine hydrochloride can be prepared in accordance with German Patent No. 159877 (GDR). In some embodiments, bendamustine or bendamustine hydrochloride is present in the pharmaceutical composition at a concentration of about 5 mg/mL to about 20 mg/mL, preferably about 8 mg/mL to about 18 mg/mL, and even more preferably at about 14.7 mg/mL.

In some embodiments, mannitol is present in the pharmaceutical composition at a concentration of about 10 mg/mL to about 30 mg/mL, preferably about 15 mg/mL to about 28 mg/mL, and more preferably at a concentration of about 25 mg/mL.

While any quality of water can theoretically be used for practicing the invention. Sterile water is preferred. This is because injection drugs are required to be sterile by FDA. In some embodiments, Sterile Water for Injection, USP, Sterile Bacteriostatic Water for Injection, USP (preserved with benzyl alcohol or parabens), and the like may be used in the preparation of the composition and in subsequent formulation steps. A person of ordinary skill in the art would understand that water is used in an amount until a desired volume is reached.

In one preferred embodiment of the pharmaceutical composition, bendamustine or bendamustine hydrochloride is present at a concentration of about 5 mg/mL to about 20 mg/mL, mannitol is present at a concentration of about 10 mg/mL to about 30 mg/mL, formic acid is present at a concentration of about 5% (v/v) to about 70% (v/v), and water constitutes the rest of the pharmaceutical composition.

In another preferred embodiment of the pharmaceutical composition, bendamustine hydrochloride is present at a concentration of about 14.7 mg/mL, and mannitol is present at a concentration of about 25 mg/mL, formic acid is present at a concentration of about 10% (v/v) to about 30% (v/v), and water constitutes the rest of the pharmaceutical composition.

The pharmaceutical composition prepared in accordance with the present invention has comparable, or even better stability than the bendamustine injectable product on the market. After being held at a temperature from about 2° C. to about 5° C. for up to 4.5 hours, it contains not more than 0.5%, preferably not more than 0.25%, and even more preferably no more than 0.16% of monohydroxy bendamustine hydrochloride (Formula II), the main degradant of bendamustine. The above impurity levels are measured by using the HPLC analysis method, as disclosed in the prior art (e.g., U.S. Pat. Nos. 8,436,190, 8,461,350, 8,609,863 and 8,791,270).

In accordance with another aspect of the invention, there is provided a lyophilized pharmaceutical composition made from the pharmaceutical composition recited above. The above liquid pharmaceutical composition (having water and formic acid) can also be called pre-lyophilization pharmaceutical composition, in order to differentiate it from lyophilized pharmaceutical composition. The term “a pre-lyophilization pharmaceutical composition” means that the composition is suitable for lyophilization but it is not required that such composition be subjected to lyophilization.

The lyophilized pharmaceutical composition is in a dry, solid form. It may present as loose powders. It contains bendamustine hydrochloride or bendamustine, and mannitol, and no water. The lyophilized pharmaceutical composition is free or substantially free of formic acid. Because formic acid is readily metabolized and eliminated by the body, even if a trace amount of formic acid is present in the lyophilized pharmaceutical composition of some embodiments, the resulting composition is still safe for medical use.

The lyophilized pharmaceutical composition has an improved impurity profile. When measured at time zero, upon reconstitution, the reconstituted composition has not more than 0.5% of monohydroxy bendamustine hydrochloride (Formula II). In some embodiments, it has not more than 0.25%, preferably no more than 0.20% of monohydroxy bendamustine hydrochloride (Formula II) at time zero. The total impurities of the reconstituted composition, measured at time zero, upon reconstitution, are not more than 1.0%, preferably not more than 0.40%, and even more preferably, not more than 0.35%. The above impurities are measured by using the HPLC analysis, substantially the same as disclosed in the prior art (e.g., U.S. Pat. Nos. 8,436,190, 8,461,350, 8,609,863 and 8,791,270).

In yet another aspect of the invention, there is provided a reconstituted pharmaceutical composition made from the lyophilized pharmaceutical composition, which in turn is made from the pre-lyophilization pharmaceutical composition recited above. The reconstituted pharmaceutical composition comprises bendamustine hydrochloride or bendamustine, mannitol, and water. It may also comprise sodium chloride or dextrose.

In a further aspect of the invention, there is provided a novel and stable process for the preparation of a bendamustine formulation with a good impurity profile. The total bendamustine degradation impurities in the final product after the process are less than 3.0%. Bendamustine in the formulation can be in the form of a free base or a pharmaceutically acceptable salt, such as an HCL salt.

The process comprises preparing a pharmaceutical composition (a.k.a., pre-lyophilization composition) comprising bendamustine or bendamustine hydrochloride, mannitol, formic acid, and water, and then subjecting the pharmaceutical composition to lyophilization.

From the process safety point of view, the process of the present invention which uses formic acid is a significant improvement over the prior art process which uses t-butanol. Formic acid, even in a concentrated form (e.g., in 85% concentration), is not flammable. In contrast, t-butanol is flammable and explosive. It has very low flash point (11° C.) and its explosive limit is as low as 2.4%-8.0%.

After bendamustine and mannitol are dissolved in formic acid and water, the resulting pre-lyophilization composition is subjected to lyophilization. In some embodiments, the pre-lyophilization composition is filtered before being lyophilized. It is discovered that a slow, stepwise, freezing-drying process is important for generating a highly porous lyophilate, which allows for good reconstitution.

According to some embodiments, the lyophilization process is conducted as follows: a pre-lyophilization composition is frozen to a temperature below −45° C., preferably from about −50° C. to about −45° C., to produce a frozen mixture; the frozen mixture is held from about −45° C. to about −40° C. for at least 3 hours, preferable at least 3.5 hours, and then subjected to a primary drying stage, which comprises applying a vacuum to reduce the pressure by an amount effective to remove water and formic acid from the frozen mixture, and while applying the vacuum, raising the temperature slowly to a primary drying temperature, wherein the primary drying temperature is from about −40° C. to about −20° C., to produce a partially dried mass. The partially dried mass is subjected to a secondary drying stage, which comprises applying a vacuum to reduce the pressure by an amount effective to further remove water and formic acid from the partially dried mass, and while applying the vacuum, slowly raising the temperature to a secondary drying temperature, wherein the secondary drying temperature is from about −20° C. to about 30° C. to produce a dry, lyophilized pharmaceutical composition containing bendamustine and mannitol.

Without wishing to be bound by theory, it is believed that the use of formic acid, as a co-solvent of water, for lyophilization contributes to the formation of a highly porous and stabilized lyophilate. Formic acid has a boiling point (100.8° C.), which is essentially the same as water (100° C.). As such, during lyophilization, it is removed from the frozen mass at a rate which is substantially the same as, water. The unsublimed or unevaporated formic acid, in its frozen stage, miscible with frozen water, continues to stabilize bendamustine HCL that is in the frozen stage, and possibly also stabilizes bendamustine HCL in a partially dried form. Because both solvents are removed at substantially the same rate, the lyophilate is formed consistently and evenly, with a high porosity. The lyophilate can therefore be reconstituted within 3-5 minutes, and the resulting reconstituted composition also exhibits improved stability.

The process for the preparation of a bendamustine formulation in accordance with the present invention may further comprise the step of reconstituting the lyophilized pharmaceutical composition (i.e., lyophilate) to form an aqueous bendamustine solution by adding water. The process may additionally comprise the step of diluting the reconstituted composition with either 0.9% Sodium Chloride Injection, USP, or 2.5% Dextrose/0.45% Sodium Chloride Injection, USP.

According to an even further aspect of the invention, there is provided a method of treating neoplastic diseases (i.e., cancers) in mammals by administering an effective amount of a pharmaceutical composition of bendamustine or bendamustine hydrochloric acid to a mammal in need thereof. The method may further comprise the steps of reconstituting the lyophilized pharmaceutical composition recited above to form an aqueous bendamustine solution for injection; and optionally diluting the aqueous bendamustine solution with either 0.9% Sodium Chloride Injection, USP, or 2.5% Dextrose/0.45% Sodium Chloride Injection, USP. The route of administration is typically by intravenous infusion over 30 to 60 minutes. The neoplastic diseases may include leukemia or Hodgkin's disease.

The invention will now be further illustrated by the following examples. It should be noticed that the present invention is not limited by the illustrative embodiments or examples. Modifications can be made without departing from the scope or spirit of the invention.

### Example 1

A method for preparing bendamustine lyophilization solution comprised the steps of: dissolving mannitol in water; adding bendamustine HCL to formic acid (in 88% aqueous solution) to form a drug solution; adding the drug solution to the mannitol solution and making up the volume to a desired level by adding water. In the resulting solution, mannitol is present at a level of about 25 mg/mL and bendamustine HCL at about 14.7 mg/mL. The formic acid concentration can vary between 5% to 70% in the resulting solution. The resulting solution is also called pre-lyophilization solution. Optionally, the pre-lyophilization solution is filtered through 0.2 micron filter and then subjected to lyophilization.

### Example 2

A method for preparing a bendamustine HCL lyophilized 25 mg/vial preparation by lyophilizing the pre-lyophilization solution prepared in accordance with Example 1 comprised the steps of: a) freezing the pre-lyophilization solution to a temperature below about −45° C., to form a frozen solution; b) holding the frozen solution at or below −40° C., preferably −45° C., for at least 300 minutes; c) ramping the frozen solution to a primary drying temperature between about −40° C. and about −25° C. to form partially dried mass by holding for about 10 to about 60 hours; d) ramping the partially dried mass to a secondary drying temperature between about −10° C. and about 30° C.; and e) holding for about 5 to about 25 hours to form a bendamustine HCL lyophilized preparation. Preferably, the lyophilization process is conducted in a vial having 25 mg of bendamustine HCL therein.

### Example 3

A method for preparing a bendamustine HCL lyophilized 100 mg/vial preparation by lyophilizing the pre-lyophilization solution prepared in accordance with Example 1 comprised the steps of: a) freezing the pre-lyophilization solution to a temperature below about −45° C., to form a frozen solution; b) holding the frozen solution at or below −40° C., preferably −45° C., for at least 300 minutes; c) ramping the frozen solution to a primary drying temperature between about −40° C. and about −25° C. to form partially dried mass by holding for about 10 to about 100 hours; d) ramping the partially mass to a secondary drying temperature between about −10° C. and about 30° C.; and e) holding for about 5 to about 35 hours to form a bendamustine HCL lyophilized preparation. Preferably, the lyophilization process is conducted in a vial having 100 mg of bendamustine HCL therein.

In Examples 2 and 3, if the lyophilization processes are conducted in final containers such as vials, the vials can be stoppered, removed and sealed to provide a finished product suitable for reconstitution. Multiple vials can be lyophilized simultaneously. Alternatively, the lyophilization may be conducted in a big container and the bendamustine HCL lyophilized powder may be dispensed into vials from the big container.

Test Results:

Three pharmaceutical compositions comprising approximately 14.7 mg/mL bendamustine HCL, and approximately 25 mg/mL mannitol concentration, formic acid, and water are prepared following the method of Example 1. The formic acid concentration in each of the pharmaceutical compositions is 10% (v/v), 20% (v/v), and 30% (v/v), respectively. The pharmaceutical compositions are held at about 2-5° C. for a few hours. In order to evaluate the stability of the compositions, samples of the compositions are taken during intervals for analysis. The level of monohydroxy bendamustine HCL, the main degradant, is analyzed by the HPLC method substantially the same as known in the art. (See U.S. Pat. Nos. 8,436,190, 8,461,350, 8,609,863 and 8,791,270).

| | | | | Hold Time |
|---|---|---|---|---|
| | Formic Acid Concentration | | | at About |
| | in Formulation (v/v) | | | 2-5° C. |
| | 10% | 20% | 30% | in hours |
| Monohydroxy | 0.06% | NA | 0.05% | Initial |
| Bendamustine | | | | (Time Zero) |
| HCL | NA | 0.18% | NA | 3.75 Hrs |
| (Impurity) | 0.24% | NA | 0.16% | 4.5 Hrs |

These pharmaceutical compositions were lyophilized as described in Example 2. The residual (i.e., lyophilized) solid was reconstituted with water for injection (USP) and analyzed for the monohydroxy bendamustine impurity at Time Zero by the known HPLC method (See U.S. Pat. Nos. 8,436,190, 8,461,350, 8,609,863 and 8,791,270). The results are shown in the table below:

| | Solid Lyophilized From a | | | |
|---|---|---|---|---|
| | Formulation That Contains | | | |
| | Formic Acid concentration (v/v) | | | |
| | 10% (v/v) | 20% (v/v) | 30% (v/v) | |
| Time Zero, upon | 0.34% | 0.19% | 0.23% | Monohydroxy |
| reconstitution | | | | Bendamustine |
| | | | | HCL Impurity |
| | 0.58% | 0.34% | 0.39% | Total Impurities |

What is claimed is:

1. A pharmaceutical composition comprising bendamustine or bendamustine hydrochloride, mannitol, formic acid, and water.

2. The pharmaceutical composition according to claim 1, wherein said formic acid is present at a concentration of about 5% (v/v) to about 70% (v/v).

3. The pharmaceutical composition according to claim 2, wherein said formic acid is present at a concentration of about 10% (v/v) to about 60% (v/v).

4. The pharmaceutical composition according to claim 1, wherein said bendamustine or bendamustine hydrochloride is present at a concentration of about 5 mg/mL to about 20 mg/mL, and said mannitol is present at a concentration of about 10 mg/mL to about 30 mg/mL.

5. The pharmaceutical composition according to claim 4, wherein said bendamustine hydrochloride is present at a concentration of about 14.7 mg/mL, and said mannitol is present at a concentration of about 25 mg/mL.

6. The pharmaceutical composition according to claim 5, wherein said formic acid is present at a concentration of about 10% (v/v) to about 30% (v/v).

7. The pharmaceutical composition according to claim 6, wherein said pharmaceutical composition, after being held at a temperature from about 2° C. to about 5° C. for about 4.5 hours, contains not more than 0.5% of monohydroxy bendamustine hydrochloride.

8. The pharmaceutical composition according to claim 7, wherein said lyophilized pharmaceutical composition contains not more than 0.25% of monohydroxy bendamustine hydrochloride.

9. A lyophilized pharmaceutical composition made from the pharmaceutical composition according to claim 1.

10. A lyophilized pharmaceutical composition made from the pharmaceutical composition according to claim 4.

11. A lyophilized pharmaceutical composition made from the pharmaceutical composition according to claim 5.

12. A lyophilized pharmaceutical composition made from the pharmaceutical composition according to claim 6.

13. The lyophilized pharmaceutical composition according to claim 9, containing not more than 0.5% of monohydroxy bendamustine hydrochloride as measured upon reconstitution of said lyophilized pharmaceutical composition with water, at time zero.

14. The lyophilized pharmaceutical composition according to claim 9 containing not more than 0.25% of monohydroxy bendamustine hydrochloride as measured upon reconstitution of said lyophilized pharmaceutical composition with water, at time zero.

15. A process for preparing a lyophilized pharmaceutical composition comprising: preparing the composition of claim 1, and lyophilizing the composition of claim 1 to obtain the lyophilized pharmaceutical composition.

16. The process for preparing a lyophilized pharmaceutical composition comprising:
freezing the composition of claim 1 to a temperature of from about −50° C. to about −45° C. to produce a frozen mixture;
holding the frozen mixture at a temperature of from about −45° C. to about −40° C. for no less than 200 minutes;
subjecting the frozen mixture to a primary drying stage, which comprises applying a vacuum to reduce the pressure by an amount effective to remove water and formic acid from the frozen mixture, and while applying the vacuum, raising the temperature to a primary drying temperature, wherein the primary drying temperature is from about −40° C. to about −25° C. to produce a partially dried mass; and
subjecting the partially dried mass to a secondary drying stage, which comprises applying a vacuum to reduce the pressure by an amount effective to further remove water and formic acid from the partially dried mass, and while applying the vacuum, raising the temperature to a secondary drying temperature, wherein the secondary drying temperature is from about −10° C. to about 30° C., to produce the lyophilized pharmaceutical composition.

17. A method of treating a neoplastic disease in mammals, comprising: reconstituting the lyophilized pharmaceutical composition of claim 9 into an aqueous bendamustine solution; and administering an effective amount of said aqueous bendamustine solution to a mammal in need thereof.

18. The method of treating neoplastic diseases in mammals according to claim 17, wherein the neoplastic disease is leukemia or Hodgkin's disease.
